(19) United States
(12) Patent Application Publication
(10) Pub. No.: US 2018/0085050 A1
(43) Pub. Date: Mar. 29, 2018

(54) SYSTEMS AND METHODS FOR INDIVIDUALIZED SLEEP OPTIMIZATION

(71) Applicants: Arizona Board of Regents on Behalf of the University of Arizona, Tucson, AZ (US); University of Pennsylvania, Philadelphia, PA (US)

(72) Inventors: Michael K. Grandner, Tucson, AZ (US); Michael Perlis, Philadelphia, PA (US)

(21) Appl. No.: 15/719,176

(22) Filed: Sep. 28, 2017

Related U.S. Application Data
(60) Provisional application No. 62/401,021, filed on Sep. 28, 2016.

(51) Int. Cl. A61B 5/00; A47C 31/00; G08B 21/06

(52) U.S. Cl. CPC A61B 5/4809; G08B 21/06; A61B 5/0022; A47C 31/008

(57) ABSTRACT

Systems and methods for sleep management are disclosed. The systems and methods, utilize one or more peripheral devices, a network, one or more networked computers, and one or more remote servers. The systems and methods are capable of collecting one or more indicators of sleep, calculating one or more sleep parameters, transmitting the sleep parameters to one or more remote servers, further calculating sleep utilization and one or more sleep recommendations using that data, and outputting one or more sleep recommendations to one or more networked computers and/or one or more peripheral devices as adjustments to sleep opportunity that can be used by the user to adjust his or her sleep cycle.

### CROSS REFERENCE TO RELATED APPLICATION(S)

This application claims priority to U.S. provisional patent application Ser. No. 62/401,021, filed on Sep. 28, 2016, which is hereby incorporated herein by reference in its entirety.

### FIELD OF THE INVENTION

The present invention relates to systems and methods for computer-implemented individualized, self-correcting, tailored systems and methods for increasing and/or optimizing sleep over a period of time using a consolidated technological platform.

### BACKGROUND OF THE INVENTION

Recent CDC data estimate that over ⅓ of US adults do not get the recommended amount of sleep to maintain optimal health and functioning. (These estimates are consistent with those from other industrialized nations as well.) This is alarming, since insufficient sleep is associated with weight gain and obesity, cardiovascular disease, diabetes, inflammation, pain, cancer, fatigue, accidents and injuries, and other adverse outcomes. This has been identified as a major unmet public health problem by the federal government, with a goal of increasing the number of adults who achieve adequate sleep identified as a national health priority in “Healthy People 2020.” However, no strategy currently exists to meet this goal. There are several barriers to achieving this. First, simply making recommendations does not change behavior. For example, telling people to quit smoking, reduce drinking, get more exercise, or reduce dietary intake is not effective in changing behavior for most people. Strategies for effective behavior change need to be developed. Second, sleep needs are difficult to quantify. Some people may need more or less sleep, and these universal recommendations do not address this. Third, ability to sleep also varies substantially from person to person, and more than this, individual sleep need substantially varies over time in relation to age, health, and performance demands. Any successful method for prescribing optimal sleep duration must be based on “the idiographic and not the nomothetic” (i.e., sleep duration must be assessed and optimized on an individual basis).

Consequently, there is a need for individualized, self-correcting, tailored systems and methods for increasing and/or optimizing sleep time over a period of time using a consolidated technological platform.

### SUMMARY OF THE INVENTION

It is therefore an object of the exemplary embodiments disclosed herein to address the disadvantages in the art and provide a sleep management system that uses networked peripheral devices to aggregate scientific data, quantifies various behavioral and physical characteristics, thereby analyzing and quantifying sleep times and/or patterns.

It is another object of the invention to have a sleep management system that utilizes quantified sleep data to determine whether and how to change sleep times and/or patterns.

It is yet another object of the invention to have a sleep management system that utilizes quantified sleep data to output recommendations in sleep times and/or patterns to users of the system.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is an exemplary embodiment of the sleep management system; and

FIG. 2 is an exemplary logic flow diagram demonstrating how the system incorporates, analyzes, and quantifies sleep data, while outputting recommendations to users.

### DETAILED DESCRIPTION OF PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the drawings.

Sleep and circadian rhythms are a key component of health and well-being in mammals. The absence of sleep can have negative effects on physical, mental, and emotional health. Too little or too much sleep can have a significant impact on cognition, cardiovascular health, the immune system, and overall health. As a consequence, monitoring and regulating sleep patterns and/or timing is important in improving the general health of individuals.

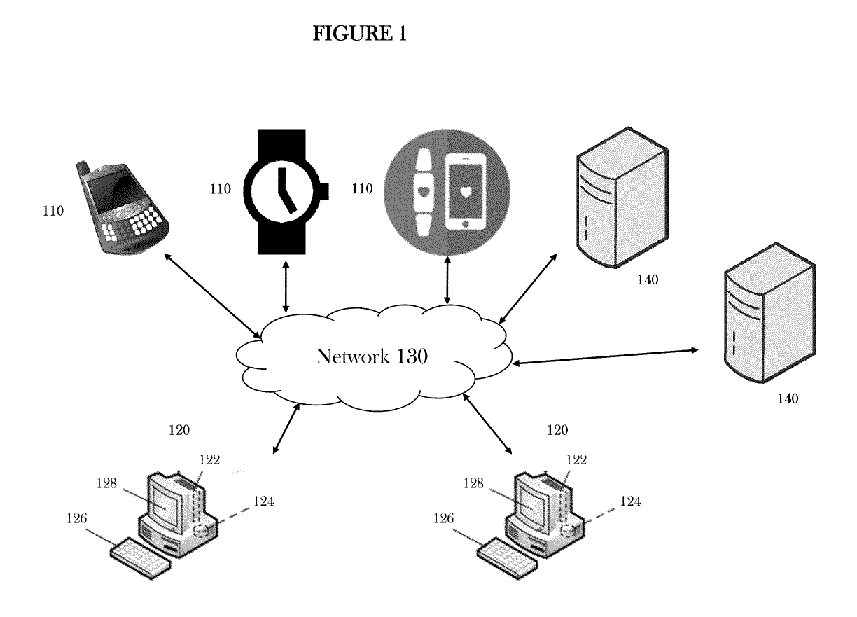

FIG. 1 is an exemplary embodiment of the sleep management system. In the exemplary system 100, one or more peripheral devices 110 are connected to one or more computers 120 through a network 130. Examples of peripheral devices 110 include clocks, smartphones, smart-clocks, tablets, wearable devices such as smartwatches, medical devices such as EKGs and blood pressure monitors, and any other devices that collect sleep data that are known in the art. The network 130 may be a wide-area network, like the Internet, or a local area network, like an intranet. Because of the network 130, the physical location of the peripheral devices 110 and the computers 120 has no effect on the functionality of the hardware and software of the invention. Both implementations are described herein, and unless specified, it is contemplated that the peripheral devices 110 and the computers 120 may be in the same or in different physical locations. Communication between the hardware of the system may be accomplished in numerous known ways, for example using network connectivity components such as a modem or Ethernet adapter. The peripheral devices 110 and the computers 120 will both include or be attached to communication equipment. Communications are contemplated as occurring through industry-standard protocols such as HTTP.

Each computer 120 is comprised of a central processing unit 122, a storage medium 124, a user-input device 126, and a display 128. Examples of computers that may be used are: commercially available personal computers, open source computing devices (e.g. Raspberry Pi), commercially available servers, and commercially available portable device (e.g. smartphones, smartwatches, tablets). In one embodiment, each of the peripheral devices 110 and each of the computers 120 of the system may have the sleep management software related to the system installed on it. In such an embodiment, sleep data may be stored locally on the networked computers 120 or alternately, on one or more remote servers 140 that are accessible to any of the networked computers 120 through a network 130. In alternate embodiments, the sleep management software runs as an application on the peripheral devices 110.

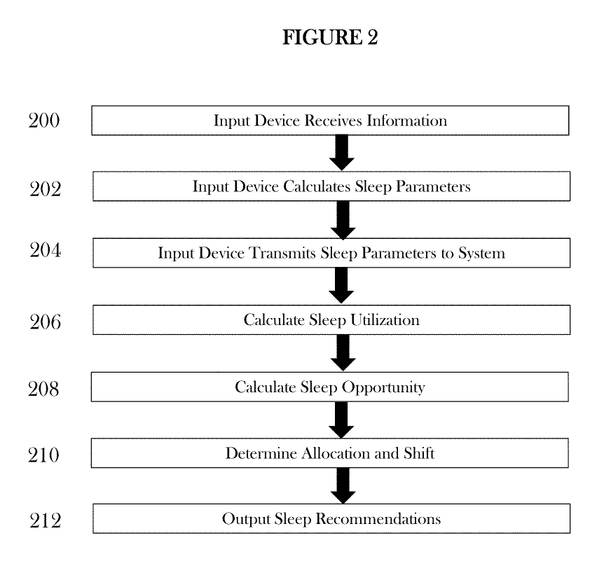

FIG. 2 is an exemplary logic flow diagram of the software processes performed using the hardware described in FIG. 1 above. In general terms, software embodiments of the invention recommend a prescribed sleep opportunity window. This software functions to optimize sleep time. The software applies the concept of sleep utilization. Sleep utilization is related to “sleep efficiency” as described in the clinical sleep research literature. Sleep utilization refers to an individual's ability to maximally utilize their time in bed. The software determines an individual's sleep utilization and then makes decisions based upon this value. Sleep utilization is determined by measuring an individual over several days, determining how much of a sleep opportunity that individual was given and how much of that opportunity was utilized for sleep. Based on these values, a proportion is calculated, reflecting the amount of sleep utilization. If this number is over a certain threshold (above a high value), the individual is given a slightly larger sleep opportunity; if this number is under a threshold (below a low value), then their sleep opportunity is actually reduced; if this number is between the high and low value, the sleep opportunity is not changed. After this decision is made, the new sleep opportunity is communicated and the process of measurement is continued. After each measurement interval, the software determines, based on sleep utilization, whether and how to change sleep opportunity.

At a high level, the invention's software performs the following algorithm: In Phase 1, the sleep opportunity and sleep ability are assessed using prospective sampling of sleep continuity data gathered via peripheral devices like actigraphs or smartwatches. Data can also be self-reported via, for example, daily sleep diaries. Self-reported data may also be concurrently gathered as both a backup method and to potentially be used as a secondary input to the software of the invention. In Phase 2, average sleep time (duration and phase) is prescribed (and represents a start point for sleep extension). The primary end goal for this phase is to regularize sleep timing and duration. Then, in Phase 3, “total time asleep,” (TST) is titrated based on how well the subject sleeps in the prescribed sleep schedule. Using sleep efficiency as the guide for weekly titration, TST is manipulated as follows: <85 SU% (where “SU%” stands for “sleep utilization percentage”), “time in bed” (TIB) is reduced by 15 minutes; 85-90% TIB remains the same; and >90% TIB is increased. The algorithm is explained in greater detail below.

The software process begins with step 200, “Input Device Receives Information,” where an input device receives data such as time spent in bed and sleep time. The input device may be a peripheral device 110 and/or a user-input device 126 in any combination. The duration of the recording period can be modified to be any number of days, with a recommendation of 3-30 days, a preferred period of 7-14 days and a proposed optimal window of 7 days (1 week). A non-exclusive list of examples of input devices follows: (1) a sleep diary where users input their values into a paper form that is scanned in and the numbers stored in a computer memory; (2) a sleep diary where users input their values into an electronic capture system and the numbers are stored in a computer memory; (3) a device that allows users to indicate (through a tap or voice command) that they are entering or exiting bed, or lying in bed awake; (4) a device that allows users to indicate (through a tap or voice command) details about their prior night's sleep, with that information stored in a computer memory; (5) a device at the bedside or attached to the bed or bedding that uses noninvasive methods to estimate when an individual is in and out of bed and/or asleep or awake; (6) a device worn by an individual that uses movement to estimate sleep and wake (e.g., accelerometer device on the head, arm, wrist, hip, or ankle, or in an article of clothing); (7) a device worn by an individual that uses biometric data (e.g., heart rate, muscle tone, breathing) to estimate sleep and wake (e.g., accelerometer device on the head, arm, wrist, hip, or ankle, or in an article of clothing); (8) a device not worn by an individual that uses movement to estimate sleep and/or wake (e.g., accelerometer or pressure transducer on the mattress or pillow); (9) a device that estimates sleep and wake using brain wave activity; (10) a device that uses motion sensing technology to estimate active time and/or time in or out of bed; and/or (11) a device that tracks ambient light to determine day/night rhythms, when lights are on/off, and/or when people are using devices with lighted screens.

The software pathway proceeds to step 202, “Input Device Calculates Sleep Parameters,” where the software performs calculations based on the data received at step 200 to derive values for a number of parameters. Exemplarily, “time in bed” (TIB) represents the total amount of time that a person was in bed, or the total time between when they first got into bed and got out of bed and can be estimated by any means. For example, it can be self-reported on a diary, estimated based on movement patterns, or estimated based on other biometric parameters. Other related parameters will include “time to bed” (TTB) and “time out of bed” (TOB). Another parameter, “total time asleep” (TST) can be estimated by any means. For example, it can be self-reported on a diary, estimated by movement patterns, or estimated by other biometric signals such as heart rate or brain signals. Other related optional parameters will include “sleep latency, or latency to fall asleep” (SL), which is measured in seconds, minutes, or hours, “time awake after initial sleep onset” (WASO), and “time spent in bed awake after the final awakening, or early morning awakenings” (EMA). “Total sleep time” would ideally be calculated by taking time in bed and subtracting these three parameters (i.e., TST=TIB−SL−WASO−EMA). Another related optional parameter may also be “number of suspected awakenings” (NWAK).

Additional optional sleep parameters may include the indication of the number of minutes each day that the observed sleep pattern deviated (DIFF). This could be represented by a value, such as the average number of minutes per day that the individual deviated from the recommended schedule. These calculations may be based on the variables DOSE, DOSE_{beginning}, and DOSE_{end}, defined below. In this case, the formula would be [(ΣDOSE−minutes)/(days)], where “minutes” refers to the number of minutes each day that the individual deviated from their prescribed schedule (DOSE) and “days” refers to the number of days that were evaluated. So if, across 7 days, the individual's actual sleep schedule deviates from DOSE by 0, 5, 10, 15, 5, 5, and 20 minutes, DIFF would be (0+5+10+15+5+8+20)/7 =9. This is just one way DIFF could be calculated. It could also be calculated such that the changes in the beginning and end of the night could be weighted. For example, if this is desired, the formula could be [(((Σ(DOSE*DOSE_{beginning})−minutes_{beginning})/(days))*DOSE_{beginning})+(((Σ(DOSE*DOSE_{end})−minutes_{end})/(days))*DOSE_{end})], where minutes_{beginning} refers to the number of minutes that differ from the amount of DOSE that should have occurred in the beginning of the night and likewise minutes_{end} refers to the number of minutes that the individual deviated from the intended DOSE that should have occurred at the end of the night. In this example, whether the intended change was focused on the beginning or end of the night will determine DIFF. If, for example, DOSE_{end} is 0, only deviations that occur in the beginning of the night will count towards the calculation of DIFF. DIFF may also weight values or consider other values in its calculation, as long as it quantifies adherence to the recommendations.

Another optional parameter would be an “indication of circadian preference” (CIRC). This variable reflects the degree to which a person's internal rhythms favor an “earlier” or “later” sleep period. This would be recorded as either “earlier” or “later” and can be measured in a number of ways. For example, it can be measured as the peak of an activity rhythm measured using accelerometry or other movement-based methods, it could be a peak level of mood or well-being measured by self-report, or it could simply be self-reported in terms of “earlier” or “later.”

Other optional parameters will reflect daytime fatigue (FATIGUE) and/or sleepiness (SLEEPY). FATIGUE represents the degree to which a person feels that they do not have the physical or mental resources to accomplish what they need to during the day. This could be self-reported or calculated based on measured parameters (e.g., activity counts, heart rate). SLEEPY represents the likelihood that an individual will fall asleep outside of the scheduled sleep time. This could be measured by self-report or calculated based on observed parameters (such as minutes of sleep time measured outside of the prescribed sleep window).

Following the calculations performed in step 202, the software pathway proceeds to step 204, “Input Device Transmits Sleep Parameters to System,” where the software passes the calculated parameters, typically a back-end server of the system on one or more remote servers 140. Useful parameters that are passed include TTB, TOB, TIB, TST. Optional parameters that are passed include SL, WASO, EMA, NWAK, DIFF, and CIRC. Additionally, the software may pass a calculated value for the fragmentation index (FI), which is calculated as FI=NWAK/TST. These parameters can be passed continuously, periodically (e.g., daily), or at the end of the recording period. The input device may also be able to compute average values for all of these parameters, but the core system, potentially located at the backend servers 140 or at peripheral devices 110, will have the functionality to compute these if needed.

Upon receipt of the sleep parameters at step 204, the system possibly at the one or more remote servers 140, at step 206, “Calculate Sleep Utilization,” performs a number of calculations to determine a sleep utilization percentage (SU%). Sleep utilization is related to “sleep efficiency” as described in the clinical sleep research literature. Sleep utilization refers to an individual's ability to maximally utilize their time in bed. Sleep utilization is determined by measuring an individual over several days, determining how much of a sleep opportunity that individual was given and how much of that opportunity was utilized for sleep. Sleep utilization can be determined through any method. This can include manually entering information into an interface, having the information passively collected by some wearable technology and exported, or having the information gathered through a method where an individual records their time in and out of bed and other sleep parameters in an external device that then exports to the system. The core formula for this calculation is: SU%=TST/TIB. In other embodiments, the system will allow for correction factors to be applied to SU% based on any of the optional parameters, as well as input device model and type (INPUT). INPUT can be a variable where a value is assigned based on input device; for example, sleep diary can be 0, actigraphy can be 1. For example, if a user wishes for a correction factor to be applied to weight accelerometry-based SU%, the system will allow for such a feature.

Incorporating the optional parameters, a consolidated formulation for SU% is: SU%=(TST/TIB)+INPUT(x₁)+TTB(x₂)+TOB(x₃)+SL(x₄)+WASO(x₅)+EMA(x₆) +FI(x₇)+DIFF(x₈)+CIRC(x₉)+FATIGUE(x₁₀)+SLEEPY(x₁₁), where the values of x₁₋₁₁ represent weights applied to each of these factors that can be determined by the user. The recommended value for these weights will be 0, but certain applications may call for this functionality. As a percentage, SU% will range from 0 to 1.

Using the sleep utilization percentage, at step 208, “Calculate Sleep Opportunity,” the system calculates and recommends a prescribed sleep opportunity window. The software determines an individual's sleep utilization percentage and then makes decisions based upon this value. Based on these values, a proportion is calculated, reflecting the amount of sleep utilization. If this number is over a certain threshold (above a high value), the individual is given a slightly larger sleep opportunity. The high threshold can take any value from 0-1.0, but it is recommended that this value be within the range of 0.85-0.95, with a proposed default optimal value of 0.90. This will be the UV. If this number is under a threshold (below a low value), then their sleep opportunity is actually reduced. The low threshold can take any value from 0-1.0, but it is recommended that this value be within the range of 0.75-0.95, with a proposed default optimal value of 0.85. This value will be the LV. If the calculated SU% is between the high and low value, the sleep opportunity is not changed. To summarize, if SU%<LV, then the recommendation will be to reduce sleep opportunity. If SU%>UV, then the recommendation will be to increase sleep opportunity. If neither case is true, then the recommendation will be to maintain sleep opportunity.

The recommendation regarding sleep opportunity is conveyed from the system exemplarily by the variable, DOSE. DOSE indicates the number of minutes by which sleep opportunity should be changed. DOSE can take any value, but it is recommended to be between 0-60 minutes and a proposed default optimal value is 15 minutes. The value of DOSE may change for each recording period but it is recommended that it remain constant. DOSE can be chosen by: (1) keeping the default value of 15 minutes; (2) specifying through an input device; (3) user-set specification of value; (4) generating a value determined based on values of DIFF (for example, DOSE could be set to be the default value minus DIFF with a lowest possible value of 0); or (5) calculating a value for DOSE based on the formula DOSE=DOSE_{default}+SU%(y₁)+INPUT(y₂)+TTB(y₃)+TOB(y₄)+SL(y₅)+WASO(y₆)+EMA(y₇)+FI(y₈)+DIFF(y₉)+CIRC(y₁₀)+FATIGUE(y₁₁)+SLEEPY(y₁₂). In this case, DOSE_{default} represents a default DOSE value, chosen a priori by any means. This value is then modified by a combination of values, where each parameter is weighted by a different value (y₁₋₁₂). These weights can be set to 0 or some other number, in order to modify the DOSE based on the values of that parameter.

At step 210, “Determine Allocation and Shift,” the system uses the DOSE value to determine the proportion of sleep to be allocated to the beginning and/or end of the sleep opportunity window, exemplarily stored as the variable ALLOC. ALLOC is exemplarily calculated as follows: The total change in sleep opportunity will be DOSE minutes added or removed from sleep opportunity. These minutes may be applied to the beginning or end of the sleep period, in any combination, as long it adds up to 100%. For example, ALLOC can be 100% at the beginning of the sleep period (earlier TTB), 100% at the end of the sleep period (later TOB), or 50% to each. The amount of sleep to be added at the beginning and/or end of the night are stored as DOSE_{beginning} and DOSE_{end} such that [DOSE_{beginning}+DOSE_{end}=1.0], DOSE_{beginning} refers to the proportion of DOSE that gets added to the beginning of the night and DOSE_{end} refers to the proportion of DOSE that gets added to the end of the night. These values can be determined based on (1) user preferences; (2) system default of DOSE_{beginning}=1.0; or (3) any combination of TIB, TST, SL, WASO, EMA, NWAK, SU%, FI, FATIGUE, SLEEPY, CIRC, and/or DIFF.

At this step, the system also determines the SHIFT, or whether to shift the sleep opportunity window. Exemplarily, a value for SHIFT_{dose} and SHIFT_{direction} will be determined. SHIFT_{dose} refers to how many minutes to shift and SHIFT_{direction} refers to whether this shift will be earlier or later. The SHIFT_{dose} values may be based on: (1) user input (e.g., a user reporting that they would prefer a shift of a specified number of minutes by answering a question such as, “How much earlier or later would you like your sleep schedule to be shifted?”); (2) input device defaults; (3) system default of SHIFT_{dose}=0; (4) system secondary default of SHIFT_{dose}=15; and/or (5) values determined by RECC (to determine SHIFT_{dose} by computing SHIFT_{dose}=(DIFF)(d), where d represents a weighting factor. Thus, if the values of SHIFT_{dose} are not defined in the system as a parameter, exemplarily, SHIFT_{dose} can be calculated as a function of DIFF. For example, if an individual is not able to adhere to recommendations, producing a value of DIFF, this can be used to determine how much of a shift is required. The weighting factor d can reflect a value to modify the impact of DIFF. One potential factor in d could be the value of SL or EMA. For example, if SL is high, it may weight SHIFT_{dose} by increasing SHIFT_{dose} if SHIFT_{direction} is 1, but reduce SHIFT_{dose} if SHIFT_{direction} is 0 or −1. SHIFT_{direction} may be based on (1) user input (e.g., a user reporting a preference for shifting earlier or later, based on the question, “Would you like your schedule to shift earlier, shift later, or not shift?”); (2) input device defaults; (3) system default of no shift (SHIFT_{direction}=0); or (4) a value determined from any combination of TIB, TST, SU%, SL, WASO, EMA, NWAK, FI, DIFF, CIRC, FATIGUE, and/or SLEEPY. SHIFT_{direction} can take the value of 0 (indicating no shift), −1 (indicating shift earlier), and 1 (indicating shift later). Exemplarily, SHIFT_{direction} could be calculated using (SL−EMA), where if (SL−EMA)<−15, SHIFT_{direction}=−1, if (SL−EMA)>15, SHICT_{direction}=1, and if −15<(SL−EMA)<15, SHIFT_{direction}=0.

At step 212, “Output Sleep Recommendations,” the system aggregates these decisions into an output sleep opportunity recommendation. After each measurement interval, the software determines, based on sleep utilization, whether and how to change the sleep opportunity. The high threshold can be anything from 80-100%, with a recommended value of 90%. The low threshold can be anything from 75-95%, with a recommended value of 85%. The amount of change to be recommended to the sleep opportunity window can vary from 1 to 60 minutes per week, with a recommended value of 15 minutes. The recording/recommendation interval can be anything from 1 to 30 days, though the recommended interval is 1 week. It is recommended that the first recording interval simply feedback with a standardized schedule without changing sleep opportunity, but this parameter can also be changed. When the software recommends a change to the sleep opportunity, this change can be reflected in either the pre-sleep period (e.g., by advancing bedtime) or the post-sleep period (e.g., by delaying waketime). The software can also change a sleep opportunity to either end of the sleep period, as long as the total magnitude change is 100% (e.g., 0% at the beginning of the night and 100% at the end of the night, 100% at the beginning of the night and 0% at the end of the night, 50%/50%, 75%/25%, etc.), though the recommended value is 100% at the beginning of the night. Feedback can also be accomplished in a number of ways. It can be a message delivered in a physical or electronic form, indicating a new prescribed sleep opportunity. It can also be delivered through an external device that either actively or passively delivers the feedback. Active delivery could include displaying a message or indicator to let a person know when their new sleep opportunity is. Passive delivery could consist of an alarm (or vibration in a wrist-worn device) for when their new bedtime and waketime would be. The output device that provides feedback to the user may or may not be the same as the input device. In this embodiment, the software delivers feedback to the output device is delivered in the form of two variables: “time to bed,” (TTB_{new}) and “time out of bed,” (TOB_{new}). These values are generally output as specific times, e.g. TTB_{new}=10:45 PM and TOB_{new}=6:00 AM. TTB_{new} is calculated as TTB_{new}=TTB−[DOSE*DOSE_{beginning}]+[SHIFT_{dose}*SHIFT_{direction}]. TOB_{new} is calculated as TOB_{new}=TOB+[DOSE*DOSE_{end}]+[SHIFT_{dose}*SHIFT_{direction}].

The feedback conveyed to the user could take several forms. For example: An email or other message specifically stating TTB_{new} and TOB_{new}, a non-verbal alert to indicate TTB_{new} and TOB_{new}, such as a vibrating alarm (in the case of a worn accelerometer), a change in light intensity or frequency (in the case of a lightbulb), a change in temperature (in the case of a thermostat). An alert may take into account values for WINDDOWN and WINDUP. WINDDOWN represents a window ranging from 5-120 minutes (default 30 minutes) where the alert occurs before TTB_{new} to allow for sufficient time to prepare for bed. For example, an alert may let you know to get ready for bed at the time [TTB_{new}−WINDDOWN] and may or may not provide an alert at TTB_{new}. For example, lights can start dimming or temperature may start cooling prior to the actual TTB_{new}. Similarly, WINDUP represents a window ranging from 5-120 minutes (default 15 minutes) prior to TOB_{new}, where a device may actually signal an alert actively or passively. For example, lights may start to brighten, temperature may start to rise, or music may start to play in anticipation of TOB_{new}. This may or may not be followed by an actual alert at TOB_{new}.

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. A sleep management system comprised of one or more peripheral devices, a network, one or more networked computers, and one or more remote servers, wherein the one or more peripheral devices and/or the networked computers collect one or more indicators of sleep, calculate one or more sleep parameters, transmit said sleep parameters to the one or more remote servers, said remote servers performing calculations to determine sleep utilization and one or more sleep recommendations, said sleep recommendations being output to one or more networked computers and/or one or more peripheral devices as adjustments to sleep opportunity.

2. The system of claim 1, wherein the sleep utilization is calculated as SU%=(TST/TIB)+INPUT(x₁)+TTB(x₂)+TOB(x₃)+SL(x₄)+WASO(x₅)+EMA(x₆)+FI(x₇)+DIFF(x₈)+CIRC(x₉)+FATIGUE(x₁₀)+SLEEPY(x₁₁), where the values of x₁₋₁₁ represent weightage values between 0 and 1.

3. The system of claim 1, wherein the system calculates a value to indicate the number of minutes by which the sleep opportunity should be changed.

4. The system of claim 3, wherein the system calculates the proportion of sleep to be allocated to the beginning and/or end of the sleep opportunity.

5. The system of claim 3, wherein the system calculates a plurality of values for shifting the sleep opportunity.

6. The system of claim 1, wherein the system outputs one or more sleep recommendations in real-time.

7. The system of claim 1, wherein the system outputs the one or more sleep recommendations in the form of a “time to bed,” (TTB_{new}) and a “time out of bed,” (TOB_{new}).

8. A method for sleep management comprising the steps of:
collecting one or more indicators of sleep;
calculating one or more sleep parameters;
transmitting the sleep parameters to the one or more remote servers;
calculating sleep utilization and one or more sleep recommendations; and
outputting one or more sleep recommendations to one or more networked computers and/or one or more peripheral devices as adjustments to sleep opportunity.

9. The method of claim 8, wherein sleep utilization is calculated as SU%=(TST/TIB)+INPUT(x₁)+TTB(x₂)+TOB(x₃)+SL(x₄)+WASO(x₅)+EMA(x₆)+FI(x₇)+DIFF(x₈)+CIRC(x₉)+FATIGUE(x₁₀)+SLEEPY(x₁₁), where the values of x₁₋₁₁ represent weightage values between 0 and 1.

10. The method of claim 8, further comprising the step of calculating a value to indicate the number of minutes by which the sleep opportunity should be changed.

11. The method of claim 10, further comprising the step of calculating the proportion of sleep to be allocated to the beginning and/or end of the sleep opportunity.

12. The method of claim 10, further comprising the step of calculating a plurality of values for shifting the sleep opportunity

13. The method of claim 8, further comprising the step of outputting the one or more sleep recommendations in real-time.

14. The method of claim 8, further comprising the step of outputting the sleep recommendations as a “time to bed,” (TTB_{new}) and a “time out of bed,” (TOB_{new}).
